(19) United States
(12) Patent Application Publication
(10) Pub. No.: US 2024/0139229 A1
(43) Pub. Date: May 2, 2024

(54) TREATMENT FOR AORTOPATHY

(71) Applicant: UNIVERSITY OF KENTUCKY RESEARCH FOUNDATION, Lexington, KY (US)

(72) Inventors: Congqing Wu, Lexington, KY (US); Hong S. Lu, Lexington, KY (US); Alan Daugherty, Lexington, KY (US); Zhenyu Li, Lexington, KY (US); Hua Li, Lexington, KY (US)

(21) Appl. No.: 18/567,315

(22) PCT Filed: Jun. 22, 2022
(86) PCT No.: PCT/US22/34590

§ 371 Date: Dec. 5, 2023

Related U.S. Application Data
(60) Provisional application No. 63/213,863, filed on Jun. 23, 2021.

(51) Int. Cl. A61K 31/7105; A61K 9/00; A61K 31/145; A61K 31/713; A61P 9/14

(52) U.S. Cl. CPC A61K 31/7105; A61K 9/0019; A61K 31/145; A61K 31/713; A61P 9/14

(57) ABSTRACT

A method of treating an aortopathy is disclosed, including identifying a subject having or at risk of developing the aortopathy and reducing the expression of and/or activity of gasdermin D (GSDMD) in the subject. The method may include administering to the subject an effective amount of a GSDMD inhibitor. The GSDMD inhibitor may reduce the expression of GSDMD in the subject and/or may reduce the activity of GSDMD in the subject.

### RELATED APPLICATIONS

This application is a utility patent application claiming the benefit of U.S. Provisional Application Ser. No. 63/213,863, filed Jun. 23, 2021, the entire disclosure of which is incorporated herein by reference.

### GOVERNMENT INTEREST

This invention was made with government support under grant number K99HL145117 awarded by the National Institutes of Health. The government has certain rights in the invention.

### TECHNICAL FIELD

The presently-disclosed subject matter generally relates to aortopathy. In particular, certain embodiments of the presently-disclosed subject matter relate to compositions and methods for use in treating, preventing, or ameliorating aortopathy.

### BACKGROUND

The aorta is the largest artery in the body, transporting blood from the heart to tissues and organs. A pathological change of the aorta is called an aortopathy. Examples of aortopathies include aneurysms (aortic dilation), dissection (tears of the aortic wall lead to blood accumulation in the aortic wall), and rupture (blood exsanguinating from the aorta). These are lethal conditions that cause more than 10,000 deaths in the United States alone each year.

Aortic aneurysms have high risk for aortic dissection and rupture that lead to more than 50% mortality.¹ Most studies report aortic diameter for the severity of this disease. However, rupture of smaller aortic aneurysms is not uncommon in humans,²⁻⁴ suggesting aneurysm size is a limited predictor of aortic rupture.

Inflammation is profound in dissected and ruptured abdominal aortic aneuryusms.^{1, 5} Inflammatory lesions often contain dead cells. Pyroptosis, distinct from apoptosis, is a lytic form of programmed cell death.^{6, 7} This process is dependent on the cleavage of gasdermin D (GSDMD) by inflammatory caspase-1 or -11.⁸⁻¹¹ Pyroptosis and production of interleukin (IL)-1β/IL-18 are the prominent outcomes of inflammasome activation in response to pathogenic molecules as well as danger signals and tissue damage.^{12, 13} Inflammasomes contribute to aortic aneurysms.^{14, 15} However, published aortic aneurysm studies of inflammasomes focused on IL-1β with conflicting results.¹⁶⁻²⁰ The clinical trial of abdominal aortic aneurysms on an IL-1β antibody (NCT02007252) was terminated prematurely due to lack of efficacy and futility.

Currently, no specific medications have been approved to prevent or treat the disease progression of aortopathies.

### SUMMARY

In accordance with the purposes and benefits described herein, novel methods for treatment of aortopathies and/or disease progression of aortopathies are described. In one aspect, a method for treating an aortopathy is described, comprising identifying a subject having or at risk of developing the aortopathy and reducing the expression of and/or activity of gasdermin D (GSDMD) in the subject. The aortopathy may include any one or combination of an aneurysm, a dissection, and a rupture.

The method includes in embodiments administering to the subject an effective amount of a GSDMD inhibitor. In embodiments, the GSDMD inhibitor reduces the expression of GSDMD in the subject. In embodiments, the inhibitor reduces the activity of GSDMD in the subject.

In embodiments, the GSDMD inhibitor comprises an antisense oligonucleotide (ASO), miRNA, siRNA, locked nucleic acid (LNA) nucleotides, or a combination thereof. The GSDMD inhibitor may comprise a guide antisense strand consisting of about 10 to about 30 nucleotides. The guide antisense strand may consist of about 10 to about 30 nucleotides that are complementary to consecutive nucleotides of the sequence of GSDMD (Accession No: NM_024736).

In embodiments, reduction in expression of and/or activity of GSDMD may be achieved using CRISPR, ASO, miRNA, siRNA, locked nucleic acid (LNA) nucleotides, or a combination thereof. In embodiments, the reduction in expression of GSDMD may be achieved using a nucleotide molecule targeting a protein coding region of the GSDMD gene. In embodiments, the reduction in expression of GSDMD may be achieved using a nucleotide molecule targeting a non-coding region of the GSDMD gene. In embodiments, the nucleotide molecule may target a promoter of the GSDMD gene. In embodiments, the nucleotide molecule may targets 3′ untranscribed regions (UTRs) of the GSDMD gene.

In embodiments, the GSDMD inhibitor may be a drug that inhibits GSDMD pore-forming activity. Suitable drugs include disulfiram and necrosulfonamide (NSA). The drug may be administered by any one or more of orally, parenterally, by injection, by subcutaneous injection, by intravenous injection, by intramuscular injection, transdermally, sublingually, topically, rectally, and intra-peritoneally.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are used, and the accompanying drawings of which:

FIGS. 1A-1D. Deficiency of Gasdermin D (GSDMD) prevents aortic rupture in mice infused with angiotensin II (AngII). Male Gsdmd^{+/+} (WT) and Gsdmd^{-/-} (KO) mice in C57BL/6J background were injected intraperitoneally with adeno-associated viral vectors (AAVs) encoding mouse PCSK9D377Y, fed a Western diet, and infused with AngII. Plasma total cholesterol concentrations were measured by an enzymatic assay kit, with results set forth in FIG. 1A. With reference to FIG. 1B, survival rate of mice were analyzed by LogRank analysis. * P<0.05. With reference to FIG. 1C, surface area was measured using an enface method. Maximal outer width were measured using an ex vivo method, with results set forth in FIG. 1D.

FIG. 2A-2C. Deficiency of gasdermin D (GSDMD) prevents aortic rupture in mice administered beta-aminopropionitrile (BAPN). Male Gsdmd^{+/+} (WT) and Gsdmd^{-/-} (KO) mice in C57BL/6J background were studied. With reference to FIG. 2A, survival rate of mice were analyzed by LogRank analysis. * P<0.05. Maximal diameter was measured on in situ images, with results shown in FIG. 2B. With reference to FIG. 2C, maximal outer widths were measured using an ex vivo method.

FIG. 3. Ex vivo images of aortas from Gsdmd^{+/+} and Gsdmd^{-/-} mice infused with AngII.

FIG. 4. Ex vivo images of aortas from Gsdmd^{+/+} and Gsdmd^{-/-} mice administered BAPN.

FIG. 5. Disulfiram inhibition of GSDMD protects against death from aortopathy in mice administered beta-aminopropionitrile (BAPN).

While the disclosure is susceptible to various modifications and alternative forms, specific embodiments thereof have been shown by way of example in the drawings and are herein described below in detail. It should be understood, however, that the description of specific embodiments is not intended to limit the disclosure to cover all modifications, equivalents and alternatives falling within the spirit and scope of the disclosure as defined by the appended claims.

### DETAILED DESCRIPTION

The details of one or more embodiments of the presently-disclosed subject matter are set forth in this document. Modifications to embodiments described in this document, and other embodiments, will be evident to those of ordinary skill in the art after a study of the information provided in this document. The information provided in this document, and particularly the specific details of the described exemplary embodiments, is provided primarily for clearness of understanding and no unnecessary limitations are to be understood therefrom. In case of conflict, the specification of this document, including definitions, will control.

The presently-disclosed subject matter includes compositions and methods for use in treating an aortopathy. As will be apparent to those of ordinary skill in the art, an aortopathy can include an aneurysm, (luminal dilation), a dissection (tear of the aortic wall lead to blood accumulation in the aortic wall), and/or a rupture (blood exsanguinating from the aorta).

As disclosed herein, it is contemplated that specific targeting of gasdermin D (GSDMD) gene expression and/or activity can have a therapeutic effect in a subject having or at risk of developing an aortopathy.

Some embodiments of the presently-disclosed subject matter include a method of treating an aortopathy, which involves identifying a subject having or at risk of developing the aortopathy; and reducing the expression of and/or activity of gasdermin D (GSDMD) in the subject.

Some embodiments of the presently-disclosed subject matter, the method involves administering to the subject a GSDMD inhibitor. In some embodiments, the GSDMD inhibitor reduces the expression of GSDMD. In some embodiments, the GSDMD inhibitor reduces the activity of GSDMD. In some embodiments, the GSDMD inhibitor prevents the activity of GSDMD.

In some embodiments, the GSDMD inhibitor comprises antisense oligonucleotide (ASO), miRNA, siRNA, locked nucleic acid (LNA) nucleotides, or a combination thereof. In other embodiments, the GSDMD inhibitor comprises a drug. In some embodiments, the GSDMD inhibitor comprises a drug that inhibits pore-forming activity of GSDMD.

In some embodiments, the GSDMD inhibitor comprises a guide antisense strand consisting of about 10, 11, 12, 13, 14, 15, 16, 17, 18, 19, 20, 21, 22, 23, 24, 25, 26, 27, 28, 29, or 30 nucleotides. In some embodiments, the guide antisense strand consisting of about 10, 11, 12, 13, 14, 15, 16, 17, 18, 19, 20, 21, 22, 23, 24, 25, 26, 27, 28, 29, or 30 nucleotides that are complementary to consecutive nucleotides of the sequence of GSDMD (Accession No: NM_024736).

In some embodiments, the guide antisense strand comprises at least eight (8) nucleotides that are guanine (G) or cytosine (C). In some embodiments, the guide antisense strand comprises at least eight (8) nucleotides that are guanine (G) or cytosine (C). In some embodiments, the guide antisense strand comprises nucleotides in which at least about 25, 30, 35, 40, 45, 50, 55, 60, 65, 70, 75, 80, 85, 90, 95, or 100% of the nucleotides are guanine (G) or cytosine (C).

In some embodiments, the guide antisense strand has a Tₘ of greater than about 45, 46, 47, 48, 49, 50, 51, 52, 53, 54, 55, 56, 57, 58, 59, 60, 61, 62, 63, 64, or 65° C. In some embodiments, the guide antisense strand has a Tₘ of greater than about 48° C.

In some embodiments of the presently-disclosed subject matter, the reduction in expression is achieved using CRISPR, ASO, miRNA, siRNA, locked nucleic acid (LNA) nucleotides, or a combination thereof.

In some embodiments of the presently-disclosed subject matter, the reduction in expression of GSDMD is achieved using a nucleotide molecule targeting a protein coding region of GSDMD gene. In some embodiments of the presently-disclosed subject matter, the reduction in expression of GSDMD is achieved using a nucleotide molecule targeting a non-coding region of GSDMD gene. In some embodiments, the nucleotide molecule targets promoter of GSDMD gene. In some embodiments, the nucleotide molecule targets 3′ untranscribed regions (UTRs) of GSDMD gene.

In some embodiments of the presently-disclosed subject matter, the reduction in activity and/or expression of GSDMD is achieved by administration of a drug in effective amounts. In some embodiments of the presently-disclosed subject matter, the reduction in activity and/or expression of GSDMD is achieved using disulfiram (Trade Name ANTABUSE; tetraethyldisulfanedicarbothioamide).

In embodiments, the drug may be administered by any pharmaceutically suitable route, including parenterally, orally, by injection (subcutaneous, intravenous, and/or intramuscular), by transdermal administration, sublingually, topically, rectally, intra-peritoneally, and others. Equally, administration in any pharmaceutically suitable form is contemplated, including as a tablet, capsule, powder that can be dispersed in a beverage, a liquid such as a solution, suspension, or emulsion that can be administered orally or by injection, a soft gel/chew capsule, a chewable bar, or any other convenient dosage form known in the art.

The terms “treatment” or “treating” refer to the medical management of a subject with the intent to cure, ameliorate, reduce, or prevent an aortopathy. As will be recognized by one of ordinary skill in the art, the term “cure” does not refer to the ability to completely remove all excess lipid accumulation in a target area. For example, in some embodiments, a cure can refer to a decrease at a level of 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 10000 decrease. Similarly, as will be recognized by one of ordinary skill in the art, the term “prevent” does not refer to an ability to completely remove any and all lipid accumulation.

Likewise, as will be recognized by one of ordinary skill in the art, the term “inhibiting” or “inhibition” does not refer to the ability to completely inactivate all target biological activity in all cases. Rather, the skilled artisan will understand that the term “inhibiting” refers to decreasing biological activity of a target, such as a GSDMD, such as can occur, for example, when a nucleotide limits the expression of the target gene, when a ligand binding site of the target protein is blocked, or when a non-native complex with the target is formed. Such decrease in biological activity can be determined relative to a control, wherein an inhibitor is not administered and/or placed in contact with the target. For example, in some embodiments, a decrease in activity relative to a control can be about a 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% decrease. The term “inhibitor” refers to a compound of composition that reduces the expression of and/or decreases the biological activity of a target, such as a GSDMD.

The terms “subject” or “subject in need thereof” refer to a target of administration, which optionally displays symptoms related to a particular disease, pathological condition, disorder, or the like. The subject of the herein disclosed methods can be a mammal. Thus, the subject of the herein disclosed methods can be a human, non-human primate, horse, pig, rabbit, dog, sheep, goat, cow, cat, guinea pig, or rodent. The term does not denote a particular age or sex. Thus, adult and newborn subjects, as well as fetuses, whether male or female, are intended to be covered. A patient refers to a subject afflicted with a disease or disorder. The term “patient” includes human and veterinary subjects. In some embodiments of the methods disclosed herein, the subject and/or patient does not have cancer.

The term “administering” refers to any method of providing a therapeutic composition to a subject. Such methods are well known to those skilled in the art and include, but are not limited to, oral administration, transdermal administration, administration by inhalation, nasal administration, topical administration, intravaginal administration, ophthalmic administration, intraaural administration, intracerebral administration, rectal administration, and parenteral administration, including injectable means such as intravenous administration, intra-arterial administration, intramuscular administration, peritoneal administration, and subcutaneous administration. Administration can be continuous or intermittent. In various aspects, a preparation can be administered therapeutically; that is, administered to treat an existing disease or condition. In further various aspects, a preparation can be administered prophylactically; that is, administered for prevention of a disease or condition.

The term “effective amount” refers to an amount that is sufficient to achieve the desired result or to have an effect on an undesired condition. For example, a “therapeutically effective amount” refers to an amount that is sufficient to achieve the desired therapeutic result or to have an effect on undesired symptoms, but is generally insufficient to cause adverse side effects. The specific therapeutically effective dose level for any particular patient will depend upon a variety of factors including the disorder being treated and the severity of the disorder; the specific composition employed; the age, body weight, general health, sex and diet of the patient; the time of administration; the route of administration; the rate of excretion of the specific compound employed; the duration of the treatment; drugs used in combination or coincidental with the specific compound employed and like factors well known in the medical arts. For example, it is well within the skill of the art to start doses of a compound at levels lower than those required to achieve the desired therapeutic effect and to gradually increase the dosage until the desired effect is achieved. If desired, the effective daily dose can be divided into multiple doses for purposes of administration. Consequently, single dose compositions can contain such amounts or submultiples thereof to make up the daily dose. The dosage can be adjusted by the individual physician in the event of any contraindications. Dosage can vary, and can be administered in one or more dose administrations daily, for one or several days.

While the terms used herein are believed to be well understood by those of ordinary skill in the art, certain definitions are set forth to facilitate explanation of the presently-disclosed subject matter.

Unless defined otherwise, all technical and scientific terms used herein have the same meaning as is commonly understood by one of skill in the art to which the invention(s) belong.

All patents, patent applications, published applications and publications, GenBank sequences, databases, websites and other published materials referred to throughout the entire disclosure herein, unless noted otherwise, are incorporated by reference in their entirety.

Where reference is made to a URL or other such identifier or address, it understood that such identifiers can change and particular information on the internet can come and go, but equivalent information can be found by searching the internet. Reference thereto evidences the availability and public dissemination of such information.

As used herein, the abbreviations for any protective groups, amino acids and other compounds, are, unless indicated otherwise, in accord with their common usage, recognized abbreviations, or the IUPAC-IUB Commission on Biochemical Nomenclature (see, Biochem. (1972) 11(9):1726-1732).

Although any methods, devices, and materials similar or equivalent to those described herein can be used in the practice or testing of the presently-disclosed subject matter, representative methods, devices, and materials are described herein.

In certain instances, nucleotides and polypeptides disclosed herein are included in publicly-available databases, such as GENBANK® and SWISSPROT. Information including sequences and other information related to such nucleotides and polypeptides included in such publicly-available databases are expressly incorporated by reference. Unless otherwise indicated or apparent the references to such publicly-available databases are references to the most recent version of the database as of the filing date of this Application.

The present application can “comprise” (open ended) or “consist essentially of” the components of the present invention as well as other ingredients or elements described herein. As used herein, “comprising” is open ended and means the elements recited, or their equivalent in structure or function, plus any other element or elements which are not recited. The terms “having” and “including” are also to be construed as open ended unless the context suggests otherwise.

Following long-standing patent law convention, the terms “a”, “an”, and “the” refer to “one or more” when used in this application, including the claims. Thus, for example, reference to “a cell” includes a plurality of such cells, and so forth.

Unless otherwise indicated, all numbers expressing quantities of ingredients, properties such as reaction conditions, and so forth used in the specification and claims are to be understood as being modified in all instances by the term “about”. Accordingly, unless indicated to the contrary, the numerical parameters set forth in this specification and claims are approximations that can vary depending upon the desired properties sought to be obtained by the presently-disclosed subject matter.

As used herein, the term “about,” when referring to a value or to an amount of mass, weight, time, volume, concentration or percentage is meant to encompass variations of in some embodiments ±20%, in some embodiments ±10%, in some embodiments ±5%, in some embodiments ±1%, in some embodiments ±0.5%, in some embodiments ±0.1%, in some embodiments ±0.01%, and in some embodiments ±0.0010% from the specified amount, as such variations are appropriate to perform the disclosed method.

As used herein, ranges can be expressed as from “about” one particular value, and/or to “about” another particular value. It is also understood that there are a number of values disclosed herein, and that each value is also herein disclosed as “about” that particular value in addition to the value itself. For example, if the value “10” is disclosed, then “about 10” is also disclosed. It is also understood that each unit between two particular units are also disclosed. For example, if 10 and 15 are disclosed, then 11, 12, 13, and 14 are also disclosed.

As used herein, “optional” or “optionally” means that the subsequently described event or circumstance does or does not occur and that the description includes instances where said event or circumstance occurs and instances where it does not. For example, an optionally variant portion means that the portion is variant or non-variant.

The presently-disclosed subject matter is further illustrated by the following specific but non-limiting examples. The following examples may include compilations of data that are representative of data gathered at various times during the course of development and experimentation related to the present invention.

### Examples

### Overview

Aortic rupture is a fatal consequence of aortic aneurysms. Macrophage infiltration is a hallmark of dissected or ruptured aortic aneurysms. Pyroptosis is one critical process in macrophage-mediated inflammation. This study determined effects of pyroptosis on aortic aneurysms, dissection, and rupture in mice with versus without GSDMD deficiency.

Studies to mimic the human aortopathies were conducted using two mouse models. One model comprises infusion of angiotensin II (an 8 amino acid peptide). Another model comprises administration of BAPN (a compound that can inhibit normal elastin or collagen formation). Both mouse models mimic many important features of the human disease.

Male Gsdmd^{+/+} and Gsdmd^{-/-} mice in C57BL/6J background (8-10 weeks old) were injected intraperitoneally with adeno-associated viral vectors encoding mouse PCSK9D377Y gain-of-function mutation and fed a Western diet to induce hypercholesterolemia. Angiotensin II (AngII, 1 μg/kg/min) was administered by infusion to these mice after two weeks of AAV injection. During the 4 weeks of AngII infusion, 5 of 13 Gsdmd^{+/+} mice died of aortic rupture, whereas no aortic rupture occurred in Gsdmd^{-/-} mice. For surviving mice, no differences of ascending or abdominal aortic dilation were detected between Gsdmd^{+/+} and Gsdmd^{-/-} mice. To determine whether protection of GSDMD deficiency on aortic rupture is mouse model specific, we subsequently examined aortic pathologies in a beta-aminopropionitrile (BAPN)-induced aortopathy mouse model. BAPN (0.5% wt/vol) was administered in drinking water to male Gsdmd^{+/+} and Gsdmd^{-/-} mice (4 weeks old) for 4 weeks. Six of 13 Gsdmd^{+/+} mice died of aortic rupture, whereas no aortic rupture occurred in Gsdmd^{-/-} mice. In mice survived, no differences of aortic diameters in ascending, arch, or abdominal aortic dilation were detected between Gsdmd^{+/+} and Gsdmd^{-/-} mice.

Animals

Gsdmd^{+/+} and Gsdmd^{-/-} mice in C57BL/6J background were bred in house.²¹ All animal experiments reported in this manuscript were performed with the approval of the University of Kentucky Institutional Animal Care and Use Committee (IACUC protocol number 2006-0009 or 2018-2968).

Adeno-Associated Viral (AAV) Vectors

AAV vectors (serotype 8) driven by a hepatocyte-specific thyroxine-binding globulin (TBG) promoter were produced by the Vector Core in the Gene Therapy Program at the University of Pennsylvania. These AAV vectors contained inserts expressing mouse PCSK9D377Y mutation (equivalent to human PCSK9D374Y gain-of-function mutation).²² For angiotensin II (AngII) infusion study, male Gsdmd^{+/+} and Gsdmd^{-/-} mice at the age of 8-10 weeks were injected intraperitoneally with AAV vectors (2×10¹¹ genome copies/mouse), and fed a Western diet (TD.88137, Envigo) for 2 weeks to induce hypercholesterolemia.

Osmotic Mini Pump Implantation and Angiotensin II (AngII) Infusion

AngII (1 μg/kg/min; Cat #H-1706; Bachem) was infused subcutaneously via mini osmotic pumps (Alzet Model #2004; Durect Corp.) for 4 weeks that was started 2 weeks of Western diet feeding. Western diet remained during AngII infusion. A surgical procedure was followed, as previously described.²³

Administration of Beta-Aminopropionitrile (BAPN)

Male and female Gsdmd^{+/+} and Gsdmd^{-/-} mice at the age of 4 weeks were administered BAPN (0.5% wt/vol; Cat #A3134; Millipore Sigma) orally in drinking water for 4 weeks.

Plasma Cholesterol Measurements

Mouse blood were collected in the presence of EDTA (final concentration: 1.8 mg/ml) by cardiac bleeding via right ventricle. Plasma total cholesterol concentrations in mice injected with AAVs encoding mouse PCSK9D377Y were measured using an enzymatic commercial kit (Cat #999-02601; Wako Chemicals USA).

Statistical Analysis

Data are represented as means±standard error of means (SEM). SigmaPlot version 14.0 (SYSTAT Software Inc.) was used for statistical analyses. Survival Log-Rank analysis was used to compare the cumulative survival rate. Incidence of aortic rupture was analyzed by Fisher exact test. Aortic diameters were analyzed by Student's t-test if data passed normality and equal variance tests. If either normality or equal variance test failed, data were analyzed by Mann-Whitney rank sum test. P<0.05 was considered statistically significant.

Results

Deficiency of GSDMD Prevents AngII-Induced Aortic Rupture

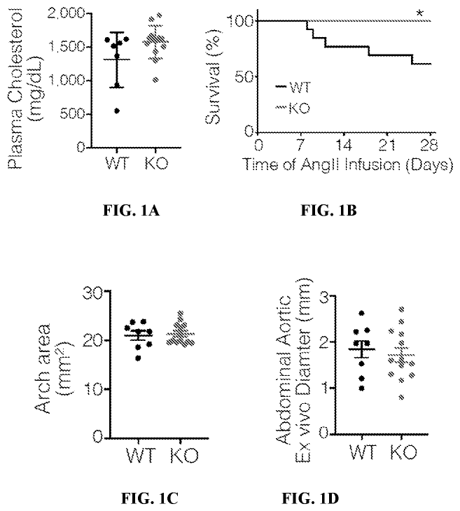

This study only used male mice because female mice have low incidence of AngII-induced aortic aneurysms.²⁴ Hypercholesterolemia augments AngII-induced aortic aneurysms.²⁵ To induce hypercholesterolemia, AAVs encoding mouse PCSK9D377Y were administered to male Gsdmd^{+/+} and Gsdmd^{-/-} mice by injection. The mice were fed a Western diet for 2 weeks prior to AngII infusion.²² Plasma total cholesterol concentrations increased rapidly within 2 weeks in both male Gsdmd^{+/+} and Gsdmd^{-/-} mice (FIG. 1A). During AngII infusion, 5 of 13 mice died due to aortic rupture in Gsdmd^{+/+} mice, but no deaths were noted in Gsdmd^{-/-} mice (FIG. 1B). After 4 weeks of AngII infusion, mice were humanely euthanized and the aortas were dissected and cleaned to measure surface area of the ascending and arch region (representing ascending and arch dilation) and outer width of the abdominal region (representing abdominal aortic dilation) since AngII induces dilations in both ascending and abdominal aortic regions (FIG. 3). Differences in surface area of the ascending aorta and maximal outer diameters of suprarenal aorta were not detected between Gsdmd^{+/+} and Gsdmd^{-/-} mice (FIGS. 1C and D).

Deficiency of GSDMD Prevents BAPN-Induced Aortic Rupture

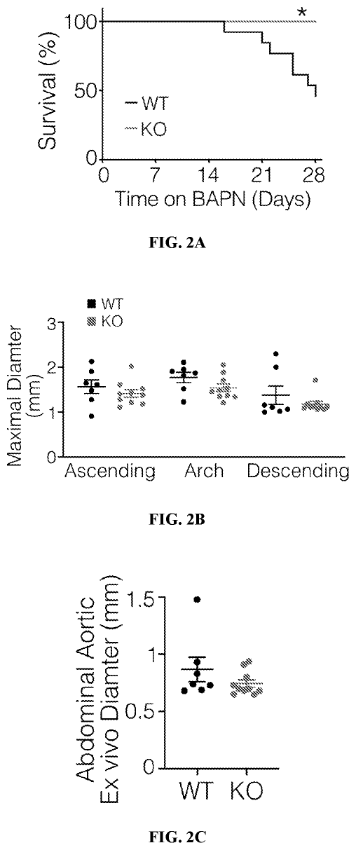

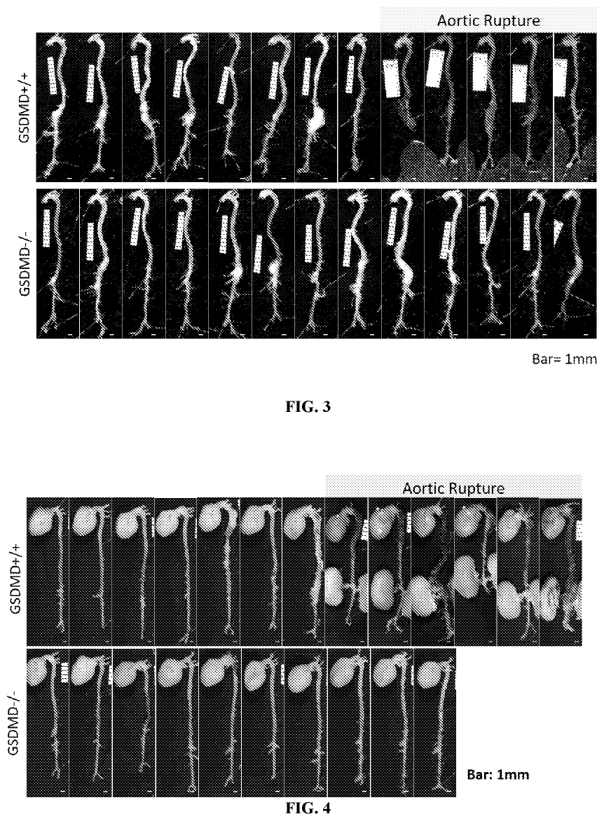

Lysyl oxidase (LOX) and LOX-like (LOXL) contribute to cross-linking of collagen and elastin of the aorta. BAPN inhibits LOX and LOXL enzymatic activity. In young mice at the age of 3-4 weeks, BAPN induces aortic dissection and rupture.²⁶ BAPN was administered to male Gsdmd^{+/+} and Gsdmd^{-/-} mice when they were 4 weeks old. Six of 13 Gsdmd^{+/+} mice died of descending aortic rupture as demonstrated by necropsy (FIG. 2A), but no Gsdmd^{-/-} mice died of aortic rupture. In mice surviving after 4 weeks of BAPN administration, aortic pathologies were found in the ascending, arch, and descending thoracic regions (FIG. 4). Ascending, arch, and descending aortic dilations were not significantly different between Gsdmd^{+/+} and Gsdmd^{-/-} mice (FIG. 2B). No apparent dilation was noted in abdominal aortas of both Gsdmd^{+/+} and Gsdmd^{-/-} mice (FIG. 2C).

In summary, deficiency of GSDMD prevented aortic rupture in mice infused with AngII or administered oral BAPN, but had no effects on aortic dilation.

Administration of Disulfiram Prevents BAPN-Induced Aortic Rupture

Having established that GSDMD deficiency prevents aortic rupture, it was desired to determine whether this could be accomplished by exogenous administration of suitable drugs. In particular, drugs affecting the pore-forming activity of GSDMD were considered. Disulfiram (tetraethyldisulfanedicarbothioamide; Trade Name ANTABUSE) is a drug used to support treatment of alcohol use disorder by producing an acute sensitivity to ethanol. Disulfiram supports this function by inhibiting the enzyme acetaldehyde dehydrogenase, providing negative reinforcement by mimicking hangover symptoms shortly after consuming alcohol. Disulfiram inhibits GSDMD pore-forming activity by modifying GSDMD. Another drug, necrosulfonamide (NSA), inhibits GSDMD pore-forming activity by directly binding to GSDMD.

For the evaluation, an animal model of aortic aneurysm as described above was used to evaluate effects of disulfiram on aortic aneurysm/aortopathy. The model comprised administration of BAPN, i.e., BAPN-induced aortopathy in a mouse model. Specifics of induction of hypercholesteremia and administration of BAPN were substantially as described supra. Male C57BL/6J mice at 3 weeks old were administered beta-aminopropionitrile (BAPN, 0.5% wt/vol) in drinking water for 28 days to induce aortic aneurysm and dissection.

The mice were separated randomly into two groups: control and treatment. For the control group, sesame oil was administered daily via intraperitoneal (I.P.) injection at 100 μL per 20 g body weight from day −1 to 27 (day −1 is the day before the first day of BAPN administration). Simultaneously, for treatment group, disulfiram at 10 mg/mL in sesame oil was administered daily via I.P. injection at 100 μL per 20 g body weight. Animal survival was monitored for 28 days during BAPN administration.

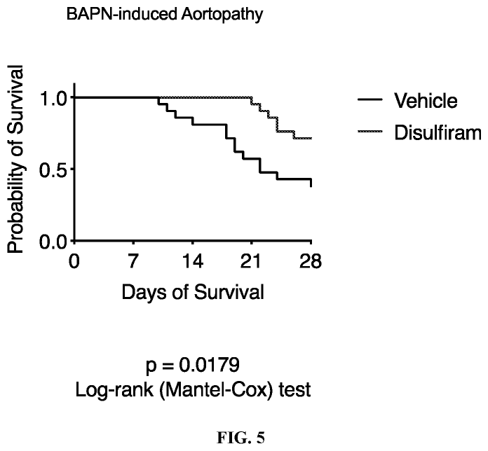

As shown in FIG. 5, administration of disulfiram provided significant positive results in survival in mice compared to administration of vehicle. Administration of disulfiram provided statistically significant increases in days of survival in the animal model of BAPN-induced aortic aneurysm and dissection.

All publications, patents, and patent applications mentioned in this specification are herein incorporated by reference to the same extent as if each individual publication, patent, or patent application was specifically and individually indicated to be incorporated by reference, including the references set forth in the following list:

### REFERENCES

1. Shen Y H, LeMaire S A, Webb N R, Cassis L A, Daugherty A and Lu H S. Aortic Aneurysms and Dissections Series. Arterioscler Thromb Vasc Biol. 2020; 40:e37-e46.
2. Satta J, Laara E and Juvonen T. Intraluminal thrombus predicts rupture of an abdominal aortic aneurysm. J Vasc Surg. 1996; 23:737-9.
3. Kazi M, Thyberg J, Religa P, Roy J, Eriksson P, Hedin U and Swedenborg J. Influence of intraluminal thrombus on structural and cellular composition of abdominal aortic aneurysm wall. J Vasc Surg. 2003; 38:1283-92.
4. Stenbaek J, Kalin B and Swedenborg J. Growth of thrombus may be a better predictor of rupture than diameter in patients with abdominal aortic aneurysms. Eur J Vasc Endovasc Surg. 2000; 20:466-9.
5. Raffort J, Lareyre F, Clément M, Hassen-Khodja R, Chinetti G and Mallat Z. Monocytes and macrophages in abdominal aortic aneurysm. Nat Rev Cardiol. 2017; 14:457-471.
6. Bergsbaken T, Fink S L and Cookson B T. Pyroptosis: host cell death and inflammation. Nat Rev Microbiol. 2009; 7:99-109.
7. Wallach D, Kang T B, Dillon C P and Green D R. Programmed necrosis in inflammation: Toward identification of the effector molecules. Science. 2016; 352:aaf2154.
8. Kayagaki N, Stowe I B, Lee B L, O'Rourke K, Anderson K, Warming S, Cuellar T, Haley B, Roose-Girma M, Phung Q T, Liu P S, Lill J R, Li H, Wu J, Kummerfeld S, Zhang J, Lee W P, Snipas S J, Salvesen G S, Morris L X, Fitzgerald L, Zhang Y, Bertram E M, Goodnow C C and Dixit V M. Caspase-11 cleaves gasdermin D for non-canonical inflammasome signalling. Nature. 2015; 526:666-71.
9. Shi J, Zhao Y, Wang K, Shi X, Wang Y, Huang H, Zhuang Y, Cai T, Wang F and Shao F. Cleavage of GSDMD by inflammatory caspases determines pyroptotic cell death. Nature. 2015; 526:660-5.
10. Ding J, Wang K, Liu W, She Y, Sun Q, Shi J, Sun H, Wang D C and Shao F. Pore-forming activity and structural autoinhibition of the gasdermin family. Nature. 2016; 535:111-6.
11. Liu X, Zhang Z, Ruan J, Pan Y, Magupalli V G, Wu H and Lieberman J. Inflammasome-activated gasdermin D causes pyroptosis by forming membrane pores. Nature. 2016; 535:153-8.
12. Lamkanfi M and Dixit V M. Mechanisms and functions of inflammasomes. Cell. 2014; 157:1013-22.
13. Latz E, Xiao T S and Stutz A. Activation and regulation of the inflammasomes. Nat Rev Immunol. 2013; 13:397-411.
14. Usui F, Shirasuna K, Kimura H, Tatsumi K, Kawashima A, Karasawa T, Yoshimura K, Aoki H, Tsutsui H, Noda T, Sagara J, Taniguchi S and Takahashi M. Inflammasome activation by mitochondrial oxidative stress in macrophages leads to the development of angiotensin II-induced aortic aneurysm. Arterioscler Thromb Vasc Biol. 2015; 35:127-36.
15. Wu D, Ren P, Zheng Y, Zhang L, Xu G, Xie W, Lloyd E E, Zhang S, Zhang Q, Curci J A, Coselli J S, Milewicz D M, Shen Y H and LeMaire S A. NLRP3 (Nucleotide Oligomerization Domain-Like Receptor Family, Pyrin Domain Containing 3)-Caspase-1 Inflammasome Degrades Contractile Proteins: Implications for Aortic Biomechanical Dysfunction and Aneurysm and Dissection Formation. Arterioscler Thromb Vasc Biol. 2017; 37:694-706.
16. Johnston W F, Salmon M, Su G, Lu G, Stone M L, Zhao Y, Owens G K, Upchurch G R, Jr. and Ailawadi G. Genetic and pharmacologic disruption of interleukin-1beta signaling inhibits experimental aortic aneurysm formation. Arterioscler Thromb Vasc Biol. 2013; 33:294-304.
17. Wakita D, Kurashima Y, Crother T R, Noval Rivas M, Lee Y, Chen S, Fury W, Bai Y, Wagner S, Li D, Lehman T, Fishbein M C, Hoffman H M, Shah P K, Shimada K and Arditi M. Role of Interleukin-1 Signaling in a Mouse Model of Kawasaki Disease-Associated Abdominal Aortic Aneurysm. Arterioscler Thromb Vasc Biol. 2016; 36:886-97.
18. Lareyre F, Clement M, Raffort J, Pohlod S, Patel M, Esposito B, Master L, Finigan A, Vandestienne M, Stergiopulos N, Taleb S, Trachet B and Mallat Z. TGFbeta (Transforming Growth Factor-beta) Blockade Induces a Human-Like Disease in a Nondissecting Mouse Model of Abdominal Aortic Aneurysm. Arterioscler Thromb Vasc Biol. 2017; 37:2171-2181.
19. Da Ros F, Carnevale R, Cifelli G, Bizzotto D, Casaburo M, Perrotta M, Carnevale L, Vinciguerra I, Fardella S, Iacobucci R, Bressan G M, Braghetta P, Lembo G and Carnevale D. Targeting Interleukin-1beta Protects from Aortic Aneurysms Induced by Disrupted Transforming Growth Factor beta Signaling. Immunity. 2017; 47:959-973 e9.
20. Batra R, Suh M K, Carson J S, Dale M A, Meisinger T M, Fitzgerald M, Opperman P J, Luo J, Pipinos, I I, Xiong W and Baxter B T. IL-1beta (Interleukin-1beta) and TNF-alpha (Tumor Necrosis Factor-alpha) Impact Abdominal Aortic Aneurysm Formation by Differential Effects on Macrophage Polarization. Arterioscler Thromb Vasc Biol. 2018; 38:457-463.
21. Wu C, Lu W, Zhang Y, Zhang G, Shi X, Hisada Y, Grover S P, Zhang X, Li L, Xiang B, Shi J, Li X A, Daugherty A, Smyth S S, Kirchhofer D, Shiroishi T, Shao F, Mackman N, Wei Y and Li Z. Inflammasome Activation Triggers Blood Clotting and Host Death through Pyroptosis. Immunity. 2019; 50:1401-1411.e4.
22. Lu H, Howatt D A, Balakrishnan A, Graham M J, Mullick A E and Daugherty A. Hypercholesterolemia induced by a PCSK9 gain-of-function mutation augments angiotensin II-induced abdominal aortic aneurysms in C57BL/6 mice. Arterioscler Thromb Vasc Biol. 2016; 36:1753-1757.
23. Lu H, Howatt D A, Balakrishnan A, Moorleghen J J, Rateri D L, Cassis L A and Daugherty A. Subcutaneous Angiotensin II Infusion using Osmotic Pumps Induces Aortic Aneurysms in Mice. J Vis Exp. 2015.
24. Robinet P, Milewicz D M, Cassis L A, Leeper N J, Lu H S and Smith J D. Consideration of Sex Differences in Design and Reporting of Experimental Arterial Pathology Studies-Statement From ATVB Council. Arterioscler Thromb Vasc Biol. 2018; 38:292-303.
25. Liu J, Lu H, Howatt D A, Balakrishnan A, Moorleghen J J, Sorci-Thomas M, Cassis L A and Daugherty A. Associations of ApoAI and ApoB-containing lipoproteins with AngII-induced abdominal aortic aneurysms in mice. Arterioscler Thromb Vasc Biol. 2015; 35:1826-1834.
26. Ren W, Liu Y, Wang X, Jia L, Piao C, Lan F and Du J. β-Aminopropionitrile monofumarate induces thoracic aortic dissection in C57BL/6 mice. Sci Rep. 2016; 6:28149.

It will be understood that various details of the presently disclosed subject matter can be changed without departing from the scope of the subject matter disclosed herein. Furthermore, the foregoing description is for the purpose of illustration only, and not for the purpose of limitation. Obvious modifications and variations are possible in light of the above teachings. All such modifications and variations are within the scope of the appended claims when interpreted in accordance with the breadth to which they are fairly, legally and equitably entitled.

1. A method of treating an aortopathy, comprising:
identifying a subject having or at risk of developing the aortopathy; and
reducing the expression of and/or activity of gasdermin D (GSDMD) in the subject.

2. The method of claim 1, comprising administering to the subject an effective amount of a GSDMD inhibitor.

3. The method of claim 2, wherein the GSDMD inhibitor reduces the expression of GSDMD in the subject.

4. The method of claim 2, wherein the GSDMD inhibitor reduces the activity of GSDMD protein in the subject.

5. The method of claim 2, wherein the GSDMD inhibitor comprises an antisense oligonucleotide (ASO), an miRNA, an siRNA, a locked nucleic acid (LNA) nucleotide, or a combination thereof.

6. The method of claim 5, wherein the GSDMD inhibitor comprises a guide antisense strand consisting of about 10 to about 30 nucleotides.

7. The method of claim 6, wherein the guide antisense strand consists of about 10 to about 30 nucleotides that are complementary to consecutive nucleotides of the sequence of GSDMD (Accession No: NM_024736).

8. The method of claim 1, wherein the reduction in expression of GSDMD is achieved using clustered regularly interspaced short palindromic repeats (CRISPR), an antisense oligonucleotide (ASO), an miRNA, an siRNA, a locked nucleic acid (LNA) nucleotide, or a combination thereof.

9. The method of claim 1, wherein the reduction in expression of GSDMD is achieved using a nucleotide molecule targeting a protein coding region of a GSDMD gene.

10. The method of claim 1, wherein the reduction in expression of GSDMD is achieved using a nucleotide molecule targeting a non-coding region of a GSDMD gene.

11. The method of claim 10, wherein the nucleotide molecule targets a promoter region of the GSDMD gene.

12. The method of claim 10, wherein the nucleotide molecule targets one or more 3′ untranscribed regions (UTRs) of the GSDMD gene.

13. The method of claim 1, wherein the aortopathy is selected from the group consisting of: an aneurysm, a dissection, and a rupture.

14. (canceled)

15. (canceled)

16. The method of claim 2, wherein the GSDMD inhibitor is a drug that inhibits GSDMD pore-forming activity.

17. The method of claim 16, wherein the drug is disulfiram.

18. The method of claim 17, wherein the disulfiram is administered by one or more routes selected from the group consisting of: of orally, parenterally, by injection, by injection, by subcutaneous injection, by intravenous injection, by intramuscular injection, transdermally, sublingually, topically, rectally, and intra-peritoneally.

19. The method of claim 18, wherein the disulfiram is administered by injection.

20. The method of claim 16, wherein the aortopathy is selected from the group consisting of: an aneurysm, a dissection, and a rupture.

21. (canceled)

22. (canceled)
